SEQUENCE LISTING (5 sequences)

SEQ ID NO: 1 - protein, 209 aa, Bos indicus
RELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 2 - protein, 630 aa, Bos indicus
GCCCGCGAGCTTGAGGAGCTGAACGTCCCCGGCGAGATCGTCGAGAGCCTCAGCAGCAGCGAGGAGAGCATCACCCGCATCAACAAGAAGATCGAGAAGTTCCAGTCCGAGGAGCAGCAGCAGACCGAGGACGAGCTTCAGGACAAGATTCACCCCTTCGCTCAGACCCAGAGCCTCGTCTACCCCTTTCCTGGCCCTATTCCTAACTCGCTGCCCCAGAACATCCCTCCGCTCACGCAGACGCCCGTCGTCGTCCCTCCGTTCCTCCAGCCTGAGGTCATGGGCGTCAGCAAGGTCAAGGAGGCCATGGCTCCCAAGCACAAGGAGATGCCCTTTCCGAAGTACCCCGTCGAGCCCTTCACCGAGAGCCAGTCGCTCACCCTCACCGACGTCGAGAACCTGCATCTGCCCCTGCCGCTCCTCCAGAGCTGGATGCACCAGCCTCACCAGCCGCTGCCTCCGACCGTCATGTTCCCTCCGCAGAGCGTCCTCAGCCTCAGCCAGAGCAAGGTCCTGCCTGTGCCTCAGAAGGCTGTCCCCTATCCTCAGCGCGATATGCCTATCCAGGCCTTCCTGCTCTACCAGGAGCCTGTTCTCGGCCCCGTGCGAGGCCCTTTTCCGATCATCGTT

SEQ ID NO: 3 - protein, 51 aa, Trichoderma reesei RUT C-30
ATGTATCGGAAGTTGGCCGTCATCTCGGCCTTCTTGGCCACAGCTCGTGCT

SEQ ID NO: 4 - DNA, 57 bp, synthetic construct
  source 1..57: mol_type = other DNA
tatccgtacgatgtccccgactacgccgctgcgtcccaccaccaccatcaccactaa

SEQ ID NO: 5 - DNA, 712 bp, synthetic construct
  source 1..712: mol_type = other DNA
cgctcgaggtcgccgtcgagaagcgccagtctgcccgcgagcttgaggagctgaacgtccccggcgagatcgtcgagagcctcagcagcagcgaggagagcatcacccgcatcaacaagaagatcgagaagttccagtccgaggagcagcagcagaccgaggacgagcttcaggacaagattcaccccttcgctcagacccagagcctcgtctacccctttcctggccctattcctaactcgctgccccagaacatccctccgctcacgcagacgcccgtcgtcgtccctccgttcctccagcctgaggtcatgggcgtcagcaaggtcaaggaggccatggctcccaagcacaaggagatgccctttccgaagtaccccgtcgagcccttcaccgagagccagtcgctcaccctcaccgacgtcgagaacctgcatctgcccctgccgctcctccagagctggatgcaccagcctcaccagccgctgcctccgaccgtcatgttccctccgcagagcgtcctcagcctcagccagagcaaggtcctgcctgtgcctcagaaggctgtcccctatcctcagcgcgatatgcctatccaggccttcctgctctaccaggagcctgttctcggccccgtgcgaggcccttttccgatcatcgttgagaacctctacttccagagccaccaccaccatcaccactaacctgcagg

(19) United States
(12) Patent Application Publication
(10) Pub. No.: US 2024/0360190 A1
(43) Pub. Date: Oct. 31, 2024

(54) RECOMBINANT VECTORS FOR EXPRESSION OF ANIMAL FREE MILK PROTEIN AND PROCESS FOR PREPARING THE ANIMAL FREE MILK PROTEIN

(71) Applicant: HENI INNOVATION PRIVATE LIMITED, Surat (IN)

(72) Inventor: Parini Kapadia, Surat (IN)

(21) Appl. No.: 18/638,496

(22) Filed: Apr. 17, 2024

(30) Foreign Application Priority Data
Apr. 17, 2023 (IN) 202321027933

(51) Int. Cl. C07K 14/47; A23C 11/08; C12N 1/14; C12N 15/80; C12Q 1/24; C12Q 1/6806; C12Q 1/686; C12Q 1/6895; C12R 1/885; G01N 33/68

(52) U.S. Cl. CPC C07K 14/4732; A23C 11/08; C12N 1/145; C12N 15/80; C12Q 1/24; C12Q 1/6806; C12Q 1/686; C12Q 1/6895; G01N 33/6803; C12R 2001/885; G01N 2333/4731

(57) ABSTRACT

The present invention broadly relates to the field of biotechnology. More particularly, the present invention relates to recombinant vector systems for the production of animal free milk proteins and a process for the production of the animal protein in recombinant host cells.

### FIELD OF THE INVENTION

The present invention broadly relates to the field of biotechnology. More particularly, the present invention relates to recombinant vector systems for the production of animal free milk proteins and a process for the production of the animal protein in in recombinant host cells.

### BACKGROUND ART

Globally, more than 7.5 billion people around the world consume milk and milk products. Demand for cow milk and dairy products is expected to keep increasing due to increased reliance on these products in developing countries as well as growth in the human population, which is expected to exceed 9 billion people by 2050.

Relying on animal agriculture to meet the growing demand for food is not a sustainable solution. According to the Food & Agriculture Organization of the United Nations, animal agriculture is responsible for 18% of all greenhouse gases, more than the entire transportation sector combined. Dairy cows alone account for 3% of this total. Various studies have identified that the dairy industry is one of the most common contributors to the greenhouse gas emissions. More particularly, it is known that dairy production is the contributor for emission of greenhouse gases such as methane, nitrous oxide and carbon dioxide. As a result, by avoiding meat and dairy products in our day-to-day diet, it can substantially reduce the impact on the planet. Further people with lactose intolerance are unable to fully digest the sugar (lactose) in natural cow milk. Lactose intolerance is nothing but the inability to break down the lactose and the reason for the same is due to humans losing ability to produce enough lactase enzyme as they age to digest and break down the lactose. The natural cow milk also contains antibiotics, hormone, cholesterol and saturated fat which may not be always good for humans.

In addition to impacting the environment, animal agriculture poses a serious risk to human health. A startling 80% of antibiotics used in the United States go towards treating animals, resulting in the development of antibiotic resistant microorganisms also known as superbugs. For years, food companies and farmers have administered antibiotics not only to sick animals, but also to healthy animals, to prevent illness. In September 2016, the United Nations announced the use of antibiotics in the food system as a crisis on par with Ebola and HIV.

It is estimated that cow milk accounts for 83% of global milk production. Accordingly, there is an urgent need for to provide bovine milk and/or essential high-quality proteins from bovine milk in a more sustainable and humane manner, instead of solely relying on animal farming.

In recent times, cellular agriculture has gained tremendous interest and has a variety of applications to address public health, environmental and animal welfare, and food security. The cellular agriculture is defined as the production of agricultural products from cell cultures rather than from whole plants or animals. The cellular agriculture is a promising technology in the food science, environmental science, nutrition, and dietetics research areas.

In the article published by Harvard Library, 90 Reasons to Consider Cellular Agriculture, disclosed that due to contamination-free production methodologies of cellular agriculture, foods can stay safer for a greater duration, ultimately granting a longer shelf life. The cellular agriculture can supply the growing demand for animal products, feeding the increasing world population of almost 10 billion by 2050. Moreover, the cellular agriculture dairy production processes may require 97% less land and 99.6% less water, may produce 65% less greenhouse gas emissions. There would be less waste of dairy products in a cellular agriculture system, since there is greater product control.

Accordingly, there is a need for the development of a synthetic process for the preparation of milk proteins which substantially reduces the greenhouse emissions that is observed in the conventional methods. The inventors of the present invention have by using genetic engineering developed recombinant vectors for the expression of animal-free milk proteins and an efficient process for preparation of the same. The dairy products containing such animal-free proteins will be safe, delicious and identical to the naturally available bovine milk and milk proteins.

### SUMMARY OF THE INVENTION

The present invention relates to recombinant vector systems for the production of animal free milk proteins and a process for the production of the animal-free milk protein in in recombinant host cells. This vector system comprises a recombinant polynucleotide sequence encoding polypeptide sequences for the target protein. Further, the present invention also provides recombinant host cells comprising the recombinant vector for the over expression of the target milk proteins.

Lastly, the present invention also provides method for the purification and isolation of the target milk protein.

### OBJECT OF THE INVENTION

An object of the present invention provides a recombinant vector system for the production of animal free milk proteins that mimic naturally occurring proteins.

Another object of the present invention provides a process for the production of the animal-free milk protein in in recombinant host cells.

Another object of the present invention provides a composition comprising the animal free milk protein along with pharmaceutically or nutraceutically acceptable carriers or vehicles.

Another object of the present invention provides a nucleic acid sequence encoding the animal free milk protein encoded by the recombinant polynucleotide sequence having 90% similarity to SEQ ID No. 2.

Another object of the present invention provides a recombinant host cell comprising recombinant vector comprising the polynucleotide sequence encoding the polypeptide sequence having 90% similarity to SEQ ID No. 1.

Another object of the present invention provides a food composition comprising an animal free milk protein having the amino acid sequence having 90% similarity to SEQ ID No. 1.

### BRIEF DESCRIPTION OF THE ACCOMPANYING FIGURES

The accompanying drawings illustrate some of the embodiments of the present disclosure and, together with the descriptions, serve to explain the disclosure. These drawings have been provided by way of illustration and not by way of limitation. The components in the drawings are not necessarily drawn to scale, emphasis instead being placed upon clearly illustrating the principles of the aspects of the embodiments.

FIG. 1—Map of Plasmid p7121B-106 for Protein Expression in Trichoderma.

FIG. 2—Map of Plasmid pZCF_Tr for Protein Expression in Trichoderma

FIG. 3—Histogram of whole plasmid sequencing

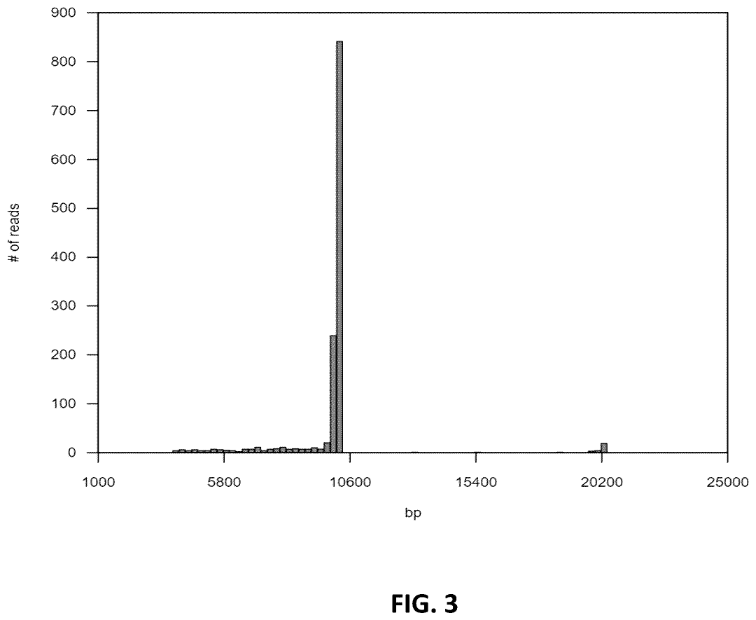

FIG. 4—SDS PAGE analysis of Trichoderma reesei RutC30 A2 Beta Casein clones and control at 120 hours. Lanes 2 through 5 Casein standards. Lane 6 is blank, Lane 7 monoconidial strain 1-C #1, Lane 8 monoconidial strain 2-B #1, Lane 9 monoconidial strain 2-E #1, Lane 10 monoconidial strain 2-J #1, Lane 11 monoconidial strain 15-A #1, Lane 12 monoconidial strain 5-B #1, Lane 13 2nd Transformation of 2-B #1 strain 2-B #2, Lane 14 2nd Transformation of 2-B #1 strain 2-B #5, Lane 15, T. reesei Rut-C30.

FIG. 5—Western blot analysis of Trichoderma reesei RutC30 A2 Beta Casein clones and control at 120 hours. Lanes 2 through 5 Casein standards. Lane 6 is blank, Lane 7 monoconidial strain 1-C #1, Lane 8 monoconidial strain 2-B #1, Lane 9 monoconidial strain 2-E #1, Lane 10 monoconidial strain 2-J #1, Lane 11 monoconidial strain 15-A #1, Lane 12 monoconidial strain 5-B #1, Lane 13 2nd Transformation of 2-B #1 strain 2-B #2, Lane 14 2nd Transformation of 2-B #1 strain 2-B #5, Lane 15, T. reesei Rut-C30.

FIG. 6—A2 Beta Casein Quantitation

### DETAILED DESCRIPTION OF THE INVENTION

At the very outset of the detailed description, it may be understood that the ensuing description only illustrates a particular form of this invention. However, such a particular form is only exemplary embodiment, and without intending to imply any limitation on the scope of this invention. Accordingly, the description is to be understood as an exemplary embodiment and teaching of invention and not intended to be taken restrictively.

Unless defined otherwise, technical and scientific terms used herein have the same meaning as commonly understood by one of ordinary skill in the art to which this invention belongs. Further, unless otherwise required by context, singular terms shall include pluralities and plural terms shall include the singular.

### Definitions

While the following terms are believed to be well understood by one of ordinary skill in the art, the following definitions are set forth to facilitate explanation of the presently disclosed subject matter.

The term “milk protein” means a protein that is found in a mammal-produced milk or a protein having a sequence that is at least 80% identical (e.g., at least 85%, at least 90%, at least 95%, at least 96%, at least 97%, at least 98%, or at least 99% identical) to the sequence of a protein that is found in a mammal-produced milk. Non-limiting examples of milk proteins include Beta-casein, Kappa-casein, Alpha-S1-casein, Alpha-S2-casein, Alpha-lactalbumin, Beta-lactoglobulin, Lactoferrin, Transferrin, and Serum albumin. Additional milk proteins are known in the art.

The term “mammal-produced milk proteins” is known in the art and means a milk protein produced by a mammal.

The term “animal-free milk proteins” is known in the art and means any milk proteins that are not produced by a mammal.

The term “genetically engineered” as used herein relates to an organism that includes exogeneous, non-native nucleic acid sequences either maintained episomally in an expression vector or integrated into the genome of the host organism.

The term “recombinant” refers to nucleic acids or proteins formed by laboratory methods of genetic recombination (e.g., molecular cloning) to bring together genetic material from multiple sources, creating sequences that would not otherwise be found in the genome. A recombinant protein is a protein created by combining sequences encoding two or more constituent proteins, such that they are expressed as a single polypeptide. Recombinant proteins may be expressed in vivo in various types of host cells, including plant cells, bacterial cells, fungal cells, mammalian cells, etc. Recombinant proteins may also be generated in vitro.

The term “recombinant” is an art known term. When referring to a nucleic acid (e.g., a gene), the term “recombinant” can be used, e.g., to describe a nucleic acid that has been removed from its naturally occurring environment, a nucleic acid that is not associated with all or a portion of a nucleic acid abutting or proximal to the nucleic acid when it is found in nature, a nucleic acid that is operatively linked to a nucleic acid which it is not linked to in nature, or a nucleic acid that does not occur in nature. The term “recombinant” can be used, e.g., to describe cloned DNA isolates, or a nucleic acid including a chemically synthesized nucleotide analog. When “recombinant” is used to describe a protein, it can refer to, e.g., a protein that is produced in a cell of a different species or type, as compared to the species or type of cell that produces the protein in nature.

As used herein, an endogenous nucleic acid sequence in the genome of an organism (or the encoded protein product of that sequence) is deemed “recombinant” herein if a heterologous sequence is placed adjacent to the endogenous nucleic acid sequence, such that the expression of this endogenous nucleic acid sequence is altered. In this context, a heterologous sequence is a sequence that is not naturally adjacent to the endogenous nucleic acid sequence, whether or not the heterologous sequence is itself endogenous (originating from the same host cell or progeny thereof) or exogenous (originating from a different host cell or progeny thereof). By way of example, a promoter sequence can be substituted (e.g., by homologous recombination) for the native promoter of a gene in the genome of a host cell, such that this gene has an altered expression pattern. This gene would now become “recombinant” because it is separated from at least some of the sequences that naturally flank it.

A nucleic acid is also considered “recombinant” if it contains any modifications that do not naturally occur to the corresponding nucleic acid in a genome. For instance, an endogenous coding sequence is considered “recombinant” if it contains an insertion, deletion, or a point mutation introduced artificially, e.g., by human intervention. A “recombinant nucleic acid” also includes a nucleic acid integrated into a host cell chromosome at a heterologous site and a nucleic acid construct present as an episome.

The term “vector” as used herein is intended to refer to a nucleic acid molecule capable of transporting another nucleic acid to which it has been linked.

One type of vector is a “plasmid,” which generally refers to a circular double stranded DNA loop into which additional DNA segments may be ligated, but also includes linear double-stranded molecules such as those resulting from amplification by the polymerase chain reaction (PCR) or from treatment of a circular plasmid with a restriction enzyme. Other vectors include cosmids, bacterial artificial chromosomes (BAC) and yeast artificial chromosomes (YAC). Another type of vector is a viral vector, wherein additional DNA segments may be ligated into the viral genome (discussed in more detail below). Certain vectors are capable of autonomous replication in a host cell into which they are introduced (e.g., vectors having an origin of replication which functions in the host cell). Other vectors can be integrated into the genome of a host cell upon introduction into the host cell and are thereby replicated along with the host genome. Moreover, certain preferred vectors are capable of directing the expression of genes to which they are operatively linked. Such vectors are referred to herein as “recombinant expression vectors” (or simply “expression vectors”).

Promoters useful for expressing the recombinant genes described herein include both constitutive and inducible/repressible promoters. Examples of inducible/repressible promoters include Cbh1 promotor or Pdc promotor. Where multiple recombinant genes are expressed in an engineered fungal, the different genes can be controlled by different promoters or by identical promoters in separate operons, or the expression of two or more genes may be controlled by a single promoter as part of an operon.

The term “operably linked” expression control sequences refer to a linkage in which the expression control sequence is contiguous with the gene of interest to control the gene of interest, as well as expression control sequences that act in trans or at a distance to control the gene of interest.

The term “expression control sequence” or “regulatory sequences” are used interchangeably and as used herein refer to polynucleotide sequences which are necessary to affect the expression of coding sequences to which they are operably linked. Expression control sequences are sequences which control the transcription, post-transcriptional events, and translation of nucleic acid sequences. Expression control sequences include appropriate transcription initiation, termination, promoter and enhancer sequences; efficient RNA processing signals, such as splicing and polyadenylation signals; sequences that stabilize cytoplasmic mRNA; sequences that enhance translation efficiency (e.g., ribosome binding sites); sequences that enhance protein stability; and when desired, sequences that enhance protein secretion. The nature of such control sequences differs depending upon the host organism; in prokaryotes, such control sequences generally include promoter, ribosomal binding site, and transcription termination sequence. The term “control sequences” is intended to include, at a minimum, all components whose presence is essential for expression, and can also include additional components whose presence is advantageous, for example, leader sequences and fusion partner sequences.

The term “transfect”, “transfection”, “transfecting,” and the like refer to the introduction of a heterologous nucleic acid into eukaryote, prokaryotic or yeast or fungal cells. “Transformation” refers to a process by which a nucleic acid is introduced into a cell, either transiently or stably. Transformation may rely on any known method for the insertion of nucleic acid sequences into a prokaryotic or eukaryotic host cell, including Agrobacterium-mediated transformation protocols, viral infection, whiskers, electroporation, heat shock, lipofection, polyethylene glycol treatment, micro-injection, and particle bombardment.

The term “recombinant host cell” (“expression host cell”, “expression host system”, “expression system” or simply “host cell”), as used herein, is intended to refer to a cell into which a recombinant vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein. A recombinant host cell may be an isolated cell or cell line grown in culture or may be a cell which resides in a living tissue or organism. Host cells can be any organism selected from the group consisting of bacteria, yeast and filamentous fungi.

The term “yeast and filamentous fungi” include, but are not limited to any Kluyveromyces sp., such as Kluyveromyces lactis, Kluyveromyces marxianus, Saccharomyces sp., such as Saccharomyces cerevisiae, Pichia sp., such as Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Hansenula polymorpha, Candida albicans, any Aspergillus sp., such as Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa.

As used herein, the term “predominantly” or variations thereof will be understood to mean, for instance, a) in the context of fats the amount of a particular fatty acid composition relative to the total amount of fatty acid composition; b) in the context of protein the amount of a particular protein composition relative to the total amount of protein composition.

The term “about,” “approximately,” or “similar to” means within an acceptable error range for the particular value as determined by one of ordinary skill in the art, which can depend in part on how the value is measured or determined, or on the limitations of the measurement system. It should be understood that all ranges and quantities described below are approximations and are not intended to limit the invention. Where ranges and numbers are used these can be approximate to include statistical ranges or measurement errors or variation. In some embodiments, for instance, measurements could be plus or minus 10%.

The phrase “essentially free of” is used to indicate the indicated component, if present, is present in an amount that does not contribute, or contributes only in a de minimus fashion, to the properties of the composition. In various embodiments, where a composition is essentially free of a particular component, the component is present in less than a functional amount. In various embodiments, the component may be present in trace amounts. Particular limits will vary depending on the nature of the component, but may be, for example, selected from less than 10% by weight, less than 9% by weight, less than 8% by weight, less than 7% by weight, less than 6% by weight, less than 5% by weight, less than 4% by weight, less than 3% by weight, less than 2% by weight, less than 1% by weight, or less than 0.5% by weight.

Unless otherwise indicated, and as an example for all sequences described herein under the general format “SEQ ID NO:”, “nucleic acid comprising SEQ ID NO: 1” refers to a nucleic acid, at least a portion of which has either (i) the sequence of SEQ ID NO:1, or (ii) a sequence complementary to SEQ ID NO:1. The choice between the two is dictated by the context.

### DETAILED DESCRIPTION

Bovine milk and dairy products have long traditions in human nutrition. Bovine milk contains the nutrients needed for growth and development of the calf, and is a resource of proteins, lipids, vitamins, amino acids and minerals. Bovine milk also contains growth factors, immunoglobulins, hormones, cytokines, peptides, nucleotides, enzymes, polyamines and other bioactive peptides. Milk proteins are important components for colour, taste and texture of the milk. Milk proteins play a very important role for the production of different kind of dairy products. Milk is consumed as a solo beverage and is also consumed in different forms like cheese, paneer, yogurt, coffee, flavoured drinks etc. Industry also has Ghee as the most consumed value-added dairy product following non-fat milk and butter.

Milk contains two types of protein Casein and Whey. Casein is the dominant protein group in bovine milk and is the major functional contributor to a family of dairy ingredients which are used ubiquitously in the food industry. The main protein found in milk is casein, especially in cow's milk. Due to casein, milk is white and opaque appearance. This protein is combined with calcium and phosphorus as clusters of casein molecules, called micelles/calcium phospho-caseinate. Casein protein consists of different proteins, Alpha-casein, Beta-casein and Kappa-casein. Casein protein is widely used for formulation of cheese, bakery products, ice-cream, infant products, coffee/tea whiteners, whipping powders etc.

Casein proteins represents a family of proteins that is present in mammal-produced milk and is capable of self-assembling with other proteins in the family to form micelles and/or precipitate out of an aqueous solution at an acidic pH. Non-limiting examples of casein proteins include Beta-casein, Kappa-casein, Alpha-S1-casein, and Alpha-S2-casein. Non-limiting examples of sequences for casein protein from different mammals are provided herein. Additional sequences for other mammalian caseins are known in the art. In the context of the invention, a “casein protein” refers to a protein at least 30%, at least 40%, at least 50%, at least 60%, at least 70%, at least 80%, at least 90%, at least 95% or completely identical to a natural mammalian casein protein. Preferred casein proteins are bovine casein proteins.

### Beta Casein

Beta-Casein is a major milk protein, representing 30% of the total protein found in cow milk, whose structure can vary depending on the breed and genetic properties of different cows. The 2 major subvariants for this protein are the A1 and A2 types, which differ by an amino acid at position 67. In the A1 type, there is a histidine amino acid in this position, whereas in the A2 type it is a proline. This difference arises from a single nucleotide difference in the $-casein gene, changing codon 67 from the more primitive CCT in the A2 gene, which codes for proline, to CAT in the A1 gene, which codes for histidine.

### A2 Beta Casein

The A2 variant is considered the oldest variant, from which the others originated via mutation. According to the National Bureau of Animal Genetic Resources (Kamal, Haryana, India), Indian dairy breeds (i.e., Gir, Tharparkar, Rathi, Red Sindhi, Sahiwal, Kankrej and Hariana) contain A2 Beta-casein. A2 Beta-casein (β-CN) comprise up to 45% of total caseins and are also phosphoproteins. A2 Beta-casein contains 5 phosphate residues, which is more hydrophobic than the other two residues and hardly soluble in water. A2 Beta-casein has 209 amino acid residues, of which 16.7% are proline evenly distributed along the polypeptide. The molecular weight of naturally occurring protein is 24.8 KDa.

### Nucleic Acids and Vectors

In an aspect, the present invention also provides nucleic acids (e.g., vectors) that include: a promoter (e.g., a yeast, bacterial, or a mammalian promoter); a sequence encoding a signal sequence; a sequence encoding a milk protein (e.g., any of the exemplary sequences described herein); and a yeast termination sequence, where the promoter is operably linked to the signal sequence, the signal sequence is operably linked to the sequence encoding the milk protein, and the terminal sequence is operably linked to the sequence encoding the milk protein. In some examples of these nucleic acids, the promoter is a constitutive promoter or an inducible promoter. Additional promoters that can be used in these nucleic acids are known in the art.

The signal sequence in any of the vectors described herein can be a signal sequence from the encoded milk protein or a different milk protein or is a signal sequence from a yeast mating factor (e.g., any alpha mating factor). In some aspects, the encoded milk protein is selected from the group of: Beta-casein (e.g., any of the Beta-casein proteins described herein), Kappa-casein (e.g., any of the Kappa-casein proteins described herein), alpha-S1-casein (e.g., any of the Alpha-S1-casein proteins described herein), Alpha-S2-casein (e.g., any of the Alpha-S2-casein proteins described herein), Alpha-lactalbumin (e.g., any of the Alpha-lactalbumin proteins described herein), Beta-lactoglobulin (e.g., any of the Beta-lactoglobulin proteins described herein), Lactoferrin (e.g., any of the lactoferrin proteins described herein), or Transferrin (e.g., any of the transferrin proteins described herein). Additional signal sequences that can be used in the present vectors are known in the art.

Any of the nucleic acids described herein can further include a bacterial origin of replication. Any of the nucleic acids described herein can further include a selection marker (e.g., an antibiotic resistance gene). The sequences of bacterial origin of replication are known in the art. non-limiting examples of antibiotic resistance genes are described herein. Additional examples of resistance genes are known in the art. non-limiting examples of termination sequences are described herein. Additional examples of termination sequences are known in the art.

In some aspects, the nucleic acids provided herein further include: an additional promoter sequence (e.g., any of the exemplary promoters described herein); an additional sequence encoding a signal sequence (e.g., any of the exemplary signal sequences described herein); a sequence encoding an additional milk protein (e.g., any of the exemplary sequences encoding a milk protein described herein); and an additional fungal termination sequence (e.g. any of the exemplary yeast termination sequences described herein), where the additional promoter sequence is operably linked to the additional sequence encoding a signal sequence, the sequence encoding the signal sequence is operably linked to the sequence encoding the additional milk protein, and the sequence encoding the additional milk protein is operably linked to the additional fungal terminal sequence. The additional milk protein can be, e.g., Beta-casein (e.g., any of the Beta-casein proteins described herein), Kappa-casein (e.g., any of the Kappa-casein proteins described herein), Alpha-S1-casein (e.g., any of the Alpha-S1-casein proteins described herein), Alpha-S2-casein (e.g., any of the Alpha-S2-casein proteins described herein), Alpha-lactalbumin (e.g., any of the Alpha-lactalbumin proteins described herein), Beta-lactoglobulin (e.g., any of the Beta-lactoglobulin proteins described herein), Lactoferrin (e.g., any of the lactoferrin proteins described herein), or Transferrin (e.g., any of the transferrin proteins described herein). In some embodiments, the nucleic acid includes a sequence encoding a Beta-casein and a sequence encoding a Kappa-casein. The promoter and the additional promoter can be the same or different. The fungal termination sequence and the additional fungal terminal sequence can be the same or different. The signal sequence and the additional signal sequence can be the same or different.

In an aspect, the present invention also encompasses a recombinant vector system containing the isolated DNA sequence encoding either casein or whey polypeptide and host cells comprising the vector. The vector may further comprise an isolated DNA sequence comprising a nucleotide sequence encoding a casein, wherein the nucleotide sequence is operably linked to a promoter, a nucleotide sequence encoding a CbhI-Cellobiohydrolase-I or a variant thereof, a nucleotide sequence encoding a bacterial resistance marker and a transcription terminator. Alternatively, the vector may further comprise an isolated DNA sequence comprising a nucleotide sequence encoding casein protein, wherein the nucleotide sequence is operably linked to a promoter, a nucleotide sequence encoding an alpha mating factor, or a variant thereof, a nucleotide sequence encoding a bacterial resistance marker and a transcription terminator. One or more of suitable promoters are utilized for expression of the genes encoding casein or whey proteins may be any promoter which is functional in the host cell and is able to elicit expression of the product encoded by the gene.

Also provided herein are examples of expression cassettes for the expression of casein or whey proteins in non-mammalian systems, such as plants and microorganisms, to produce recombinant casein proteins. The expression cassette may comprise, for example, a promoter, a 5′ untranslated region (UTR), a sequence encoding one or more casein proteins, and a terminator. The expression cassette may further comprise a selectable marker and retention signal.

In some aspect, a nucleic acid comprises a sequence encoding a target protein. In some embodiments, a nucleic acid comprises a sequence encoding a target protein, which is operably linked to a promoter. In another aspect, a nucleic acid comprises, in order from 5′ to 3′, a promoter, a 5′ untranslated region (UTR), a sequence encoding a milk protein, and a terminator.

Introducing Nucleic Acids into a Cell

Methods of introducing nucleic acids (e.g., any of the nucleic acids described herein) into a cell to generate a host cell are well-known in the art. Non-limiting examples of techniques that can be used to introduce a nucleic acid into a cell include: calcium phosphate transfection, dendrimer transfection, liposome transfection (e.g., cationic liposome transfection), cationic polymer transfection, electroporation, cell squeezing, sonoporation, optical transfection, protoplast fusion, impalefection, hydrodynamic delivery, gene gun, magnetofection, and viral transduction.

One skilled in the art would be able to select one or more suitable techniques for introducing the nucleic acids into a cell based on the knowledge in the art that certain techniques for introducing a nucleic acid into a cell work better for different types of host cells. Exemplary methods for introducing a nucleic acid into a yeast cell are described in Kawai et al., Bioeng. Bugs 1:395-403, 2010.

### Host Cells

In an aspect, the present invention provides host cells including any of the nucleic acids (e.g., vectors) described herein. In another aspect, the nucleic acid described herein is stably integrated within the genome (e.g., a chromosome) of the host cell. In other aspects, the nucleic acid described herein is not stably integrated within the genome of the host cell.

In some aspects of the present invention, the host cell is a yeast strain or a bacterial strain. an organism selected from the group consisting of bacteria, yeast and filamentous fungi. In a preferred aspect, the host cell can be, e.g., a yeast strain selected from the group of: a Kluyveromyces sp., Pichia sp., Saccharomyces sp., Tetrahymena sp., Yarrowia sp., Hansenula sp., B lastobotrys sp., Candida sp., Zygosaccharomyces sp., Trichoderma sp., and Debaryomyces sp. Additional non-limiting examples of yeast strains that can be used as the host cell are Kluyveromyces lactis, Kluyveromyces marxianus, Saccharomyces cerevisiae, and Pichia pastoris. Additional species of yeast strains that can be used as host cells are known in the art.

In another aspect, the host cell can be a protozoa, such as, e.g., Tetrahymena thermophile, T. hegewischi, T. hyperangularis, T. malaccensis, T. pigmentosa, T pyriformis, and T. vorax.

In another aspect, the present invention provides a method for isolating the animal-free milk proteins components by recombinantly expressing them in any of the host cells provided herein.

### EMBODIMENTS OF THE PRESENT INVENTION

An important embodiment of the present invention is to provide a recombinant vector system for the production of animal free milk proteins comprising a polynucleotide sequence encoding a polypeptide sequence encoding the animal free milk protein selected from the group comprising of Beta-casein, Kappa-casein, Alpha-S1-casein, Alpha-S2-casein, Alpha-lactalbumin, Beta-lactoglobulin, Lactoferrin, and/or Transferrin, a nutritional selection marker polynucleotide sequence operably linked to a promoter, a nucleotide sequence encoding a ‘HIS’ tag, a nucleotide sequence encoding a bacterial resistance marker and a transcription terminator.

A preferred embodiment of the present invention provides that the animal free milk protein is Beta-casein.

A preferred embodiment of the present invention provides that the animal free milk protein is A2 Beta-casein.

A preferred embodiment of the present invention provides that the animal free milk protein is encoded by a polypeptide sequence having 90% similarity to SEQ ID No. 1.

Yet another important embodiment of the present invention provides that the recombinant vector comprises:
a. a nucleotide sequence encoding a promoter selected from T. reesei cellobiohydrolase 1 (cbh1) promoter, or T. reesei Ppdc1 promoter or, T. reesei PamdS promoter, or T. reesei Psdh promoter;
b. a nucleotide sequence encoding signal peptide selected from CBH1 signal peptide, or SPcbh1 signal peptide;
c. a nucleotide sequence encoding CBH1 core coding sequence,
d. a nucleotide sequence encoding a flexible linker,
e. a nucleotide sequence encoding a Kex2 protease recognition/cleavage site,
f. a nucleotide sequence encoding an animal free milk protein having 90% similarity to SEQ ID No. 2,
g. a nucleotide sequence encoding a ‘HIS’ tag;
h. a nucleotide sequence encoding a terminator sequence selected from T. viride cbh1 terminator, or Trichoderma reesei Tcbh1 terminator, or Trichoderma reesei Tpdc1 terminator, or Trichoderma reesei TamdS terminator, or Trichoderma reesei Tsdh terminator; and
i. a nucleotide sequence encoding an acetamidase gene (amdS), or succinate Dehydrogenase Subunit Genes (sdh), or an hygromycin phosphotransferase (hph) gene.

An important preferred embodiment of the present invention provides that the recombinant vector system comprises:
a. a nucleotide sequence encoding T. reesei cellobiohydrolase 1 (cbh1) promoter,
b. a nucleotide sequence encoding CBH1 signal peptide,
c. a nucleotide sequence encoding CBH1 core coding sequence,
d. a nucleotide sequence encoding a flexible linker,
e. a nucleotide sequence encoding a Kex2 protease recognition/cleavage site,
f. nucleotide sequence encoding an animal free milk protein having 90% similarity to SEQ ID No. 2,
g. a nucleotide sequence encoding a ‘HIS’ tag;
h. a nucleotide sequence encoding T. viride cbh1 terminator, and
i. a nucleotide sequence encoding an acetamidase gene (amdS).

A preferred embodiment of the present invention provides that the animal-free milk protein mimics the naturally occurring milk proteins.

A preferred embodiment of the present invention provides that the animal-free milk protein is obtained after codon optimization.

An important embodiment of the present invention provides a method for the production of animal-free milk proteins in recombinant host cells, the method comprising the steps of:
i. designing plasmid expression vectors comprising a polynucleotide sequence encoding a polypeptide sequence encoding the animal free milk protein having 90% similarity to SEQ ID No. 1 operably linked to promoter by synthesis of gene segments and multiplication of gene segments using PCR techniques;
ii. carrying spore isolation and protoplastation of fungal host cell to obtain the competent protoplast;
iii. combining the recombinant vector with the competent protoplast obtained in step (ii) on ice followed by incubating the nutrient media plate for 2-4 weeks; and
iv. isolating the sporulating clones obtained in step (iii) by analysing the genomic DNA to identify the overexpressing milk protein gene;
v. inoculating the positive transformed clones identified in step (iv) in PDB media in baffled conical flask and growing them in a shaker at 150-180 rpm at 28-30° C. for 5-7 days;
vi. collecting the supernatant from the samples cultured in step (v) every 24 hr time interval centrifugating at 5000-8000 rpm for 5 min at 2-4° C. to harvest the animal free milk proteins;
vii. analysing the supernatant using SDS-PAGE and western blot to confirm the overexpression of beta-casein, followed by isolation and purification of the animal free milk protein.

A preferred embodiment of the present invention provides that step (ii) of the method comprises the following steps:
a. adding double autoclaved Milli-Q water in the Trichoderma reesei RUT-C30 plate or slant to rinse the slant/plate surface to release the spores from the mycelium;
b. filtering the suspension obtained in step (a) through autoclaved miracloth in the laminar to collect filtrate containing spores or conidia;
c. washing the spore suspension by centrifuge at 5000-8000 rpm and 1-5 mins and discarding the supernatant followed by washing spore pellet with Milli-Q water and centrifuging at 5000 rpm for 3 min,
d. repeating this washing step 3 times and re-suspending the spore pellet in autoclaved Milli-Q water;
e. inoculating spore suspension in PDB and incubating at 25° C. to 28° C. with 100 to 200 rpm for 5 to 6 hrs followed by confirming the spore germination under microscope to carry out protoplastation;
f. centrifuging the spore suspension obtained in step (e) at 5000-8000 rpm and 3-5 mins to obtain the pellet and discarding the supernatant;
g. adding SMC to the pellet and centrifuging at 5000-8000 rpm and 5-8 mins, repeated this step four times;
h. incubating the spore pellet with lysing enzymes for 30 min-1 hour with regular gentle tapping/mixing till protoplastation is shown on 60% population,
i. adding SMC solution to the suspension and centrifuging at 2000-8000 rpm and 3-5 mins and discarding the supernatant, this step is repeated thrice with SMC;
j. Wash the pellet obtained in step (i) with STC buffer and centrifuging at 2000-8000 rpm and 3-5 mins and discard supernatant followed by dissolving the pellet in STC to obtain the competent protoplast.

A preferred embodiment of the present invention provides that step (iv) of the method comprises the following steps:
a. Growing transformed host cells in MMS-N+Acetamide liquid media at 28-30° C. for 5 to 10 days under constant shaking conditions;
b. centrifuging the culture in a tube at 12000×g to 18000×g for 15-20 mins followed by discarding the supernatant using a pipette and collecting the cell pellet and freezing it using Liquid N2¬ for storing at −80° C. to crush the genomic DNA in the cell pellets in liquid N2¬;
c. adding lysis buffer to crushed cell pellet followed by subjecting the samples to vortex several times and adding Phenol:Chloroform:Isoamyl alcohol mixture to vortexed sample;
d. centrifuging the lysed cell suspension obtained in step (c) at 12000×g to 18000×g for 15-20 min at room temperature and collecting the upper aqueous layer in a microcentrifuge tube, and adding to it, 100% ethanol followed by mixing the resulting solution by inverting the tube several times;
e. centrifuging the solution obtained in step (d) at 12000×g to 18000×g for 5 to 10 min at room temperature, discarding the supernatant and drying the DNA pellet at 37° C. incubator;
f. washing the dried DNA pellet with 70% Ethanol and centrifugating at 12000×g to 18000×g for 5-10 min followed by drying the resulting pellet at 37° C. and resuspended in elution buffer supplemented with of RNaseA and incubating in a 50-60° C. heat block for 10-20 min to obtain the genomic DNA.

A preferred embodiment of the present invention provides that the host cell is selected from bacterial, yeast or fungal cells.

A preferred embodiment of the present invention provides that the host cell is a fungal cell.

A preferred embodiment of the present invention provides that the host cell is Trichoderma reseei.

Another important embodiment of the present invention provides a composition comprising the animal free milk protein along with pharmaceutically or nutraceutically acceptable carriers or vehicles.

Another important embodiment of the present invention provides a nucleic acid sequence encoding the recombinant fusion protein encoded by the recombinant polynucleotide sequence having 90% similarity to SEQ ID No. 2.

Another important embodiment of the present invention provides a recombinant host cell comprising recombinant vector comprising the polynucleotide sequence encoding the polypeptide sequence having 90% similarity to SEQ ID No. 1.

A preferred embodiment of the present invention provides that the host cell is selected from bacterial, plant, yeast or fungal cells.

A preferred embodiment of the present invention provides that the host cell is a fungal cell.

Another important embodiment of the present invention provides a food composition comprising an animal free milk protein having the amino acid sequence having 90% similarity to SEQ ID No. 1, wherein the food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, dairy substitutes, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.

### Advantages

The present invention demonstrates the following advantages:
animal friendly,
reduces greenhouse gas emissions,
cost effective,
increased yield of the proteins
more nutritional value (up to 99% pure protein)
free of lactose, antibiotic, saturated fat, cholesterol, pathogen etc

Without limiting the scope of the present invention as described above in any way, the present invention has been further explained through the examples provided below.

### EXPERIMENTAL DATA

### Example 1: Identification and Codon Optimization of the Gene Sequences in Bos Indicus

High quality milk producing cow such as Gir cattle (Bos indicus) was identified. Thereafter, further genetic analysis of the identified Gir cattle to identify the specific genetic segment which is responsible for production of desired milk proteins was performed.

In-silico data analysis and codon optimization of identified gene sequences was carried out to obtain the recombinant polynucleotide sequence encoding the target milk protein.

### Example 2: Preparation of the Recombinant Plasmid 1 for the Expression of the Target Milk Protein

The inventors of the present invention have further designed the expression vector and primers for gene cloning procedure. Novel expression vector was prepared by combining the different properties and sites of native expression vectors used for Trichoderma reesei. Multi-copy integration of the target genes into the genome of Trichoderma reesei was the most efficient strategy. This vector design helped to achieve multiple integration and the inventors have performed modification for antibiotic selection marker site for easy selection of positive transformants at higher concentration of antibiotic.

Further, different promoter and secretary peptides were prepared in designing the constructs for expression of codon optimized gene sequences. The recombinant plasmid was obtained by synthesis of gene segments and multiplication of gene segments using PCR techniques. Plasmids were constructed in vitro by digesting (cutting) DNA fragments with restriction enzymes at specific sites (restriction sites) and then ligating (joining) the resulting fragments. The constructed DNA is usually amplified in E. coli to analyze its structure.

Plasmid Design-1 for A2 Beta Casein Expression in T. reesei:

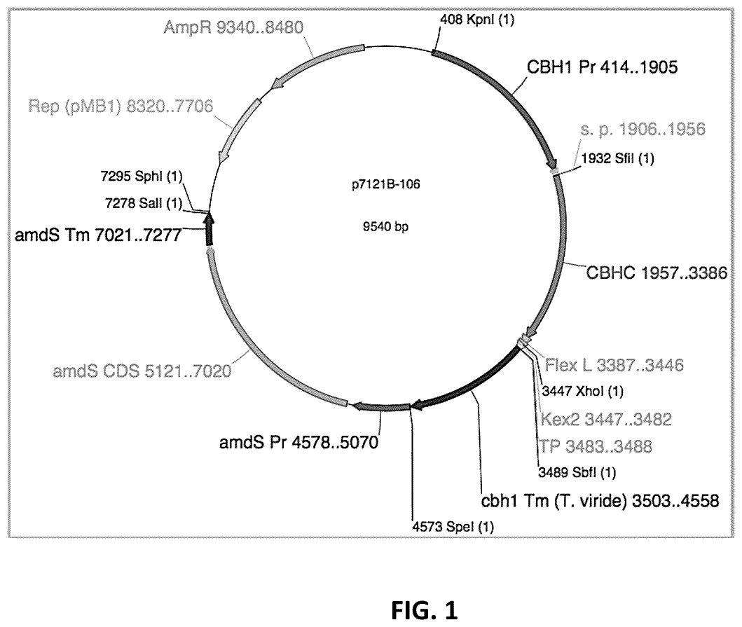

Protein expression in Trichoderma was carried out using plasmid p7121B-106, a pUC57-based plasmid (FIG. 1). It contains a replication origin and an ampicillin resistance marker for manipulation in Escherichia coli. The plasmid was designed by cloning between the KpnI and SalI restriction sites the sequences for Trichoderma transformation and protein expression. The plasmid designed so that the plasmid contained from 5′-3′ direction: T. reesei cellobiohydrolase 1 (cbh1) promoter, CBH1 signal peptide, CBH1 core coding sequence including two introns, a flexible linker, a Kex2 protease recognition/cleavage site, nucleotide sequence encoding the A2-Beta casein milk protein codon optimized in example 1, T. viride cbh1 terminator, and an acetamidase gene (amdS) as a nutritional selection marker.

The inventors chose acetamidase gene as it allows transformants to use acetamide as the sole nitrogen source in minimal medium. The A2-Beta Casein gene placed between XhoI (5′) and SbfI (3′) sites can be replaced with another gene of interest by restriction digestion and ligation. BTR vector p7121B-106 contains an XhoI site that is followed by a Kex2 cleavage site between the CBH1 core sequence and a POI gene in the vector. Pro-A2 Beta Casein sequence was codon-optimized according to T. reesei codon bias and synthesized as an XhoI-SbfI fragment that contains the Kex2 cleavage site sequence added to the N-terminus of Pro-TG.

### Example 3: Preparation of the Recombinant Plasmid 2 for the Expression of the Target Milk Protein

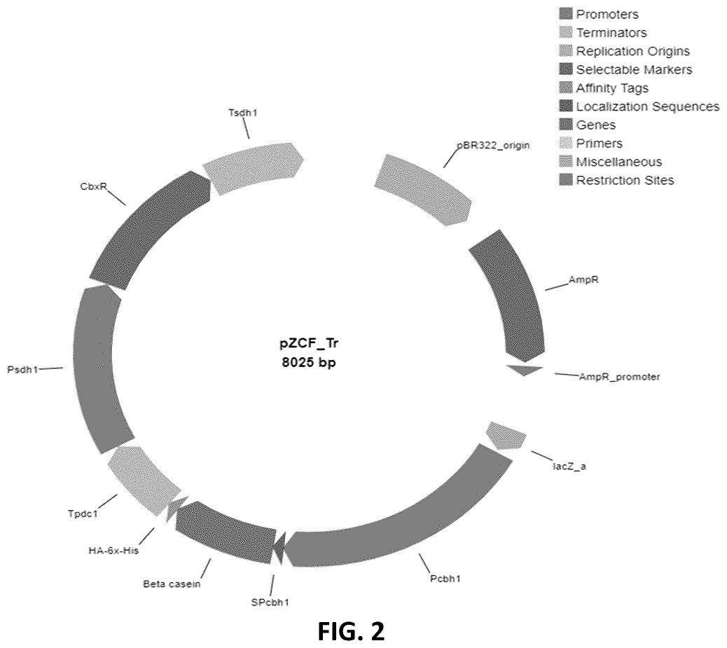

Protein expression in Trichoderma was carried out using plasmid pZCF_Tr, a pUC57-based plasmid (FIG. 2). The standard pZCF_Tr plasmid used to transform Trichoderma reesei RUT-C30 contains amdS or mutated succinate dehydrogenase 1 (sdh1) genes as a selection marker. The codon optimized amdS gene from Aspergillus nidulans codes for acetamidase. Positive clones of Trichoderma reesei RUT-C30 will be able to utilize acetamide as a sole nitrogen source in case of amdS marker and will exhibit a resistant phenotype to carboxine, a fungicide (in case of mutated sdh1 marker). The gene encoding milk proteins (kappa casein, beta casein (a2 variant) and beta-lactoglobulin) are driven by Trichoderma reesei RUT-C30 native promoters that are inducible by different carbon sources. A representative plasmid pZCF_Tr_0005, which has T.

The plasmid variants containing different promoters and selection markers were assembled using NEB HiFi DNA assembly kit and the assembled products were transformed into DH10B E. coli competent cells using heat shock method. The resultant transformed colonies were selected on LB agar plates with ampicillin (100 ug/ml). The selected clones were screened using colony PCR using gene specific primers (primer list), restriction digestion and whole plasmid sequencing. The sequencing results confirmed the correct plasmid assembly. The following histogram figure represents the whole plasmid sequencing where the number of reads is plotted against the size in base pairs (bp). A single sharp increase in number of reads at the expected plasmid size indicates positive assembly.

The XhoI-SbfI fragment encompassing the Kex2 cleavage site, Pro-A2 Beta Casein sequence was isolated from the synthetic gene plasmid received from GeneArt. The fragment was cloned at the XhoI and SbfI sites in p7121B106 to create construct A2 beta casein.

### Example 4: Transfecting the Recombinant Plasmid to Host Cells

Recombinant plasmid was developed to express the target milk protein A2 Beta casein.

The transformation of recombinant plasmid into Trichoderma reesei was carried out by electroporation. Further, screening of positive transformants using PCR and nucleotide sequencing was carried out.

### Materials and Methods:

Glucose (50%): For 1 L: Boil 500 mL Milli-Q (MQ) in a 1000 mL beaker on a heated magnetic stirrer. Slowly add 500 g of D (+)-glucose anhydrous. After glucose has been dissolved, let the solution cool down to RT, add MQ up to 1 L and autoclave.
ASPA+N (50×): For 1 L: Add 297.5 g NaNO3, 26.1 g KCl and 74.8 g KH 2 PO 4 to 600 mL MQ in a 1 L cylinder. When all salts are dissolved, set pH to 5.5 with KOH (use 5 M KOH). Add MQ up to 1 L and autoclave.
ASPA−N (50×): For 1 L: Add 26.1 and 74.8 g KH 2 PO 4 to 600 mL MQ in a 1 L cylinder. When dissolved, set pH to 5.5 with KOH. Add MQ up to 1 L and autoclave.
MgSO 4 (1 M): For 1 L: Add 246.5 g MgSO 4-7H 2 O to 600 mL MQ in a 1 L cylinder. When all salts are dissolved, add MQ up to 1 L and autoclave.
SMC: For 1 L: Add 242.3 g D-sorbitol, 5.5 g CaCl2) and 3.9 g MES hydrate to 600 mL MQ in a 1 L cylinder. When everything is dissolved, set pH to 7.2 using 1 M NaOH and 1 M HCl. Add MQ up to 1 L and autoclave.
TC: For 1 L: Add 5.5 g CaCl 2 and 1.2 g Tris to 800 mL MQ in a 1 L cylinder. When everything is dissolved, set pH to 5.8 using 1 M NaOH and 1 M HCl. Add MQ up to 1 L and autoclave.
STC: For 1 liter: Add 242.3 g D-sorbitol to 600 mL TC in a 1 L cylinder. When sorbitol is dissolved, add TC up to 1 L and autoclave.
PEG buffer: For 10 mL: Add 2.5 g of Polyethylene glycol 6000 (PEG) to a 50-mL tube, add TC up to 10 mL under sterile conditions and dissolve PEG by shaking. Use PEG solution only fresh.
Minimal medium+sucrose+agar (MMS): For 500 mL: Dissolve 162.6 g of D (+)-Sucrose in 480 mL of MQ, add 6 g (1.2%) of agar and autoclave (see Note 9). For pyrG selection, add after autoclaving under sterile conditions: 10 mL of 50×ASPA+N, 1 mL of 1 M MgSO4, 500 m L of 1,000× trace element solution. For hygromycin selection, add after autoclaving under sterile conditions: 10 mL of 50×ASPA+N, 1 mL of 1 M MgSO4, 500 m L of 1,000× trace element solution, 5 mL of 50 mg/mL caffeine and 1 mL of 100 mg/mL hygromycin (see Note 10). For amdS selection, add after autoclaving under sterile conditions: 10 mL of 50×ASPA−N, 5 mL of 1 M acetamide, 5 mL of 1.5 M CsCl, 1 mL of 1 M MgSO 4 and 500 m L of 1,000× trace element solution.
MMS top agar: For 500 mL: Dissolve 162.6 g of D (+)-saccharose in 480 mL of MQ, add 3 g of agar (final concentration is 0.6%) and autoclave (see Note 9). Store the top agar at 65° C. and add under sterile conditions: 10 mL of 50×ASPA+N, 1 mL of 1 M MgSO4, 500 m L of 1,000× trace element solution. Before use, transfer the top agar to a 47-50° C. water bath.
Trace element solution (1,000×): For 1 L: Add 10 g EDTA, 4.4 g ZnSO 4-7H 2 O, 1.01 g MnCl 2 4H 2 O, 0.32 g CoCl 2·6H 2 O, 0.315 g CuSO 4·5H 2 O, 0.22 g (NH4) 6 Mo 7 O24·4H 2O, 1.11 g CaCl 2 and 1.0 g FeSO 4·7H 2 O to 600 mL MQ. When dissolved, set pH to 4.0 with 1 M NaOH and 1 M HCl, fill MQ up to 1 L and autoclave
Acetamide (1 M): For 100 mL: Add 5.91 g of acetamide to 50 mL of warm MQ (about 50-60° C.) in a 100 mL cylinder. When acetamide is dissolved, add MQ up to 100 mL, sterilize by filtration and store at 4° C. The final concentration in cultivation medium is 10 mM (see Note 3).

Note: For carboxine resistance selection, the selection media should contain carboxine (100 ug/ml).

i. Preparation of the Recombinant Plasmids Obtained in Example 2-3:

The recombinantly designed plasmids prepared in examples 2-3 were digested with KpnI and SalI to isolate an 8.0-Kb fragment from the smaller 2.6-Kb plasmid backbone to obtain 10 ug of linearized plasmid. The linearized larger fragment encompassing the expression cassette and amdS selection marker from A2 beta casein was purified through agarose gel electrophoresis using the QIAGEN Gel Purification Kit. Aliquots of 1.0 and 2.0 μL of a 10-fold diluted sample were analysed on an agarose gel with Thermo Fisher Low Mass Ladder to check purity and estimate concentration. The linear plasmid containing the required expression cassette was concentrated to 500 ng/uL concentration. Typically, 5-10 μg of purified DNA in a volume equal to 20 μL was used in the protoplast transformation.

ii. Spore Isolation of Trichoderma reesei

The inventors added 5 ml double autoclaved Milli-Q water in the Trichoderma reesei RUT-C30 plate or slant. The slant/plate surface were rinsed so that spores are released from the mycelium.

Filter this suspension through autoclaved miracloth in the laminar and collect filtrate (contains spores or conidia). Wash the spore suspension by centrifuge at 5000 rpm for 3 min. Discard the supernatant and wash spore pellet with Milli-Q water 3 times by using centrifuge at 5000 rpm for 3 min. Re-suspend spore pellet in around 1 ml of autoclaved Milli-Q water.

Take 7-8 μl spore suspension on slide and observe it under microscope. Store the spore suspension by using 20% glycerol in −80° C.

iii. Protoplast Generation of Trichoderma reesei

Protoplasts were prepared and transformed by following the protocols described in the paper by Punt and van den Hondell (Methods Enzymol. 1992, 216:447-457).

1 ml of spore suspension 1×108/ml was innoculated in 100 ml of PDB and incubate at 28° C. with 180 rpm for 8 h. After 8 hours, spore germination was observed under microscope and after confirming the germination, use it for protoplastation. 100 ml of culture was pelleted in 50 ml falcon at 5000 rpm for 3 min and discarding the supernatant in laminar. 10 ml of SMC was added to the pellet and centrifuge at 5000 rpm for 5 min. This step was repeated once again.

The spore pellet was incubated with lysing enzymes Yatalase in SMC buffer 10 mg/ml, enough buffer was added to re-suspend the pellet, for 30 min-1 hour with regular gentle tapping/mixing. The incubated suspension was observed under microscope after 30 min interval. When around 60% population protoplastation was seen, 10 ml SMC solution was added to the suspension and centrifuge at 2000 rpm for 5 min. The supernatant was discarded. Repeat step 9 thrice with 10 ml of SMC.

The pellet was then washed with 10 ml STC buffer with centrifuge at 2000 rpm for 5 min and the supernatant was discarded. The pellet was dissolved in 2 ml of STC to make 15-20 aliquots of 100 μl each and stored in −80° C. deep freezer for further use. (*if liquid nitrogen is available, treat 4 aliquots with liquid nitrogen and compare their viability with aliquots directly stored at −80° C.).

iv. Transformation of the Protoplast with the Recombinant Vectors

### a) Material and Methods

Transformation plate: Plate preparation for MMS-AA agar plate (Minimal media+sucrose+agar) (*AMDS selection) wherein the 1st layer is MMS-ASPA−N (15 ml), with acetamide (final conc. 0.25 M). Let it cool and settle as precipitate. Add 1 ml PEG 6000 with above transformation mixture at 25° C. for 5 minutes (room temperature or incubator). Add 2 ml STC. 2nd layer is MMS-ASPA+N (5 ml) with above made transformant mixture (3.125 ml, added at 40° C.). Pour on 1st layer and let it settle. (Be extra cautious about temperature at which transformant mixture is being added in the media)

Control plate: First control plate is same as transformation plate without linearized plasmid. Pour and plate only protoplast. Second control plate is only MMS-ASPA−N (20 ml). Pour and plate only protoplast. Third control plate is only MMS-ASPA+N (20 ml). Pour and plate only protoplast.

Glucose (50%): For 1 L: Boil 500 mL Milli-Q (MQ) in a 1000 mL beaker on a heated magnetic stirrer. Slowly add 500 g of D (+)-glucose anhydrous. After glucose has been dissolved, let the solution cool down to RT, add MQ up to 1 L and autoclave.

ASPA+N (50×): For 1 L: Add 297.5 g NaNO3, 26.1 g KCl and 74.8 g KH 2 PO 4 to 600 mL MQ in a 1 L cylinder. When all salts are dissolved, set pH to 5.5 with KOH (use 5 M KOH). Add MQ up to 1 L and autoclave.

ASPA−N (50×): For 1 L: Add 26.1 and 74.8 g KH 2 PO 4 to 600 mL MQ in a 1 L cylinder. When dissolved, set pH to 5.5 with KOH. Add MQ up to 1 L and autoclave.

MgSO 4 (1 M): For 1 L: Add 246.5 g MgSO 4-7H 2 O to 600 mL MQ in a 1 L cylinder. When all salts are dissolved, add MQ up to 1 L and autoclave.

SMC: For 1 L: Add 242.3 g D-sorbitol, 5.5 g CaCl2 and 3.9 g MES hydrate to 600 mL MQ in a 1 L cylinder. When everything is dissolved, set pH to 7.2 using 1 M NaOH and 1 M HCl. Add MQ up to 1 L and autoclave.

TC: For 1 L: Add 5.5 g CaCl 2 and 1.2 g Tris to 800 mL MQ in a 1 L cylinder. When everything is dissolved, set pH to 5.8 using 1 M NaOH and 1 M HCl. Add MQ up to 1 L and autoclave.

STC: For 1 liter: Add 242.3 g D-sorbitol to 600 mL TC in a 1 L cylinder. When sorbitol is dissolved, add TC up to 1 L and autoclave.

PEG buffer: For 10 mL: Add 2.5 g of Polyethylene glycol 6000 (PEG) to a 50-mL tube, add TC up to 10 mL under sterile conditions and dissolve PEG by shaking. Use PEG solution only fresh.

Minimal medium+sucrose+agar (MMS): For 500 mL: Dissolve 162.6 g of D (+)-Sucrose in 480 mL of MQ, add 6 g (1.2%) of agar and autoclave (see Note 9). For pyrG selection, add after autoclaving under sterile conditions: 10 mL of 50×ASPA+N, 1 mL of 1 M MgSO4, 500 m L of 1,000× trace element solution. For hygromycin selection, add after autoclaving under sterile conditions: 10 mL of 50×ASPA+N, 1 mL of 1 M MgSO4, 500 m L of 1,000× trace element solution, 5 mL of 50 mg/mL caffeine and 1 mL of 100 mg/mL hygromycin (see Note 10). For amdS selection, add after autoclaving under sterile conditions: 10 mL of 50×ASPA−N, 5 mL of 1 M acetamide, 5 mL of 1.5 M CsCl, 1 mL of 1 M MgSO 4 and 500 m L of 1,000× trace element solution.

MMS top agar: For 500 mL: Dissolve 162.6 g of D (+)-saccharose in 480 mL of MQ, add 3 g of agar (final concentration is 0.6%) and autoclave (see Note 9). Store the top agar at 65° C. and add under sterile conditions: 10 mL of 50×ASPA+N, 1 mL of 1 M MgSO4, 500 m L of 1,000× trace element solution. Before use, transfer the top agar to a 47-50° C. water bath.

Trace element solution (1,000×): For 1 L: Add 10 g EDTA, 4.4 g ZnSO 4-7H 2 O, 1.01 g MnCl 2·4H 2 O, 0.32 g CoCl 2·6H 2 O, 0.315 g CuSO 4·5H 2 O, 0.22 g (NH4) 6 Mo 7 O24·4H 2O, 1.11 g CaCl 2 and 1.0 g FeSO 4·7H 2 O to 600 mL MQ. When dissolved, set pH to 4.0 with 1 M NaOH and 1 M HCl, fill MQ up to 1 L and autoclave

Acetamide (1 M): For 100 mL: Add 5.91 g of acetamide to 50 mL of warm MQ (about 50-60° C.) in a 100 mL cylinder. When acetamide is dissolved, add MQ up to 100 mL, sterilize by filtration and store at 4° C. The final concentration in cultivation medium is 10 mM (see Note 3).

For carboxine resistance selection, the selection media should contain carboxine (100 ug/ml).

### b) Transformation Process:

Protoplasts were transformed with approximately ˜10 μg DNA of the A2 beta casein fragment in a total volume of 20 μL. Transformation was carried out by mixing and incubate 10 μg single digested plasmid with 100 μl protoplast and 25 μl PEG buffer on ice for 20 minutes. The solution was further mixed with 10 mL Trich MM Regeneration overlay (0.7%) containing 10 mM Acetamide as the sole nitrogen source. Samples were poured over Trich MM Regeneration plates without Ammonium Sulphate and incubated at 28-30° C. for 4-5 days. Seventy-five colonies were isolated from transformation with the construct A2 Beta Casein and streaked for purification on Trich MM(−N)+10 mM Acetamide as the sole nitrogen source. Plates were incubated again for 2-3 days at 28° C.

Isolated colonies from each original transformant clone were inoculated into 25 mL of Inoculum Medium in a 125-mL baffled flask and incubated for 3 days at 28° C. A small volume of the inoculum culture (2.5 mL) was used to inoculate 50 mL Production Medium in a 250-mL baffled flask. Flasks were grown for 5 days with daily sampling at 28° C., 170 rpm. The production medium contains cellulose, which is an inducer for expression of the gene driven by the cbh1 promoter.

The cultures in production flasks were sampled by removing ˜1.5 mL of culture to a 1.5-mL Eppendorf tube and centrifuged for 10 min at 13,000 rpm (17,900×g) in the Eppendorf centrifuge 5417C. The supernatant was removed to a new 1.5-mL Eppendorf tube and frozen at −20° C. for analysis of secreted protein. Typically, two samples were saved from each flask. One for analysis and the second as back up.

The supernatants from the production flasks were tested for expression of A2 Beta Casein.

### Example 5: Expression and Isolation of the Target Milk Protein in Transformed Host Cells

i. Selection and Screening of Transformants by Genomic DNA Analysis

The individual sporulating clones obtained in example 5 were picked after 4 weeks and streaked on to fresh plates containing corresponding selection media. Two vials were made from each flask by mixing 1.0 mL inoculum culture+1.0 mL 30% glycerol and frozen down. A few drops from each inoculum flask were also plated on PD agar plates and incubated for 2 weeks at 28° C. to sporulation. The plates were incubated at 28° C. for 5-7 days. Transformant colonies will start to sporulate by this time. Spores were harvested and saved for isolation of monoconidial clones from the best primary transformants. After 2 weeks, individual sporulating clones were picked and plated on to the centre of the selection plate. This step is critical to make sure the transformants are mono-conodial isolates. The mono-conodial isolates are inoculated in to 5 ml fresh PDB media and grown for 4-5 days. The cultures were spun down 5000 rpm for 5 min and the cell pellets were stored at −80 deg C. until further use.

The frozen pellets were processed for genomic DNA isolation according to the following protocol:
To isolate genomic DNA from different Trichoderma strains the respective strains of Trichoderma were grown in MMS-N+Acetamide liquid media (table 1) at 28° C. for 5 days under constant shaking conditions.
Centrifuge the culture in a 15 ml tube at 12000×g for 15 mins. Remove the supernatant using a pipette.
The cell pellet was collected and frozen using Liquid N2¬ and cell pellets were stored at −80° C.
The genomic DNA was isolated from the cell pellets by crushing it in liquid N2¬.
Take 500 μl (w/v) of crushed cell pellet and add 500 μl of lysis buffer.
Then vortex the sample several times.
Then add 500 μl of Phenol:Chloroform:Isoamyl alcohol mixture.
The lysed cell suspension was centrifuged at 16000×g for 15 min at room temperature.
Following centrifugation, the upper 400/more μl aqueous layer was collected in a 1.5 ml microcentrifuge tube, and to it, 1 ml of 100% molecular biology grade ethanol (Merck) was added, and the resulting solution was mixed by inverting the tube several times.
The solution was centrifuged at 16000×g for 5 min at room temperature. After this discard the supernatant and dry the DNA pellet at 37° C. incubator.
The dried DNA pellet is further washed with 475 μl 70% molecular biology grade Ethanol at 16000×g for 5 min.
The resulting pellet is further dried at 37° C. and resuspended in 40 μl elution buffer supplemented with 100 μg/ml of RNaseA and incubated in a 50° C. heat block for 10 min.
Genomic DNA thus isolated were run on agarose gels to check for the integrity and finally stored at −20° C. refrigerator for future use.
The isolated genomic DNA was used as template for PCR to confirm the presence of the transgene, which in this case is beta-casein.
   ii. Expression Confirmation Using SDS-PAGE and Western Blot Analysis.

The clones that are verified positive with the genomic DNA PCR were inoculated in 50 ml PDB media in 250 ml baffled conical flask. The cultures were grown at 180 rpm at 28° C. for 5-7 days. Supernatant samples from the culture were collected in every 24-hr time interval and harvested at 5000 rpm for 5 min at 4° C. The supernatants were analysed using SDS-PAGE and western blot to confirm the overexpression of beta-casein. The protocols that were used are as followed:

### SDS-PAGE

Sample preparation: The target milk proteins to be analysed are extracted from cells or tissues and denatured by boiling them in an SDS-containing buffer. The denatured proteins are then loaded onto the gel.
Gel preparation: The polyacrylamide gel is prepared by mixing acrylamide and bis-acrylamide monomers, a cross-linker, and a catalyst. The mixture is poured into a mold, and a comb is inserted to create wells where the protein samples will be loaded.
Electrophoresis: The gel is placed in an electrophoresis chamber filled with a buffer solution that conducts electricity. The gel is then submerged in the buffer, and an electric field is applied. The proteins migrate through the gel according to their size and charge.
Staining and visualization: After electrophoresis, the proteins can be visualized by staining them with a dye, such as Coomassie blue. The stained gel is then destained, and the protein bands are visible as distinct bands.

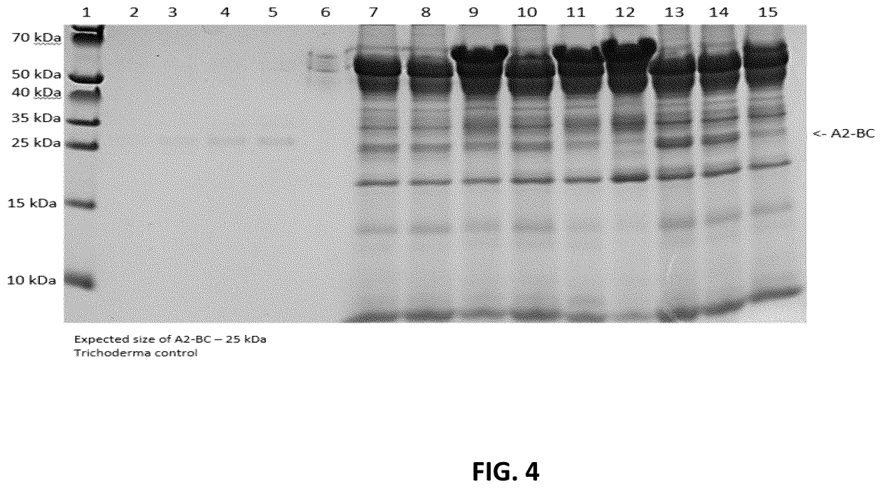

FIG. 4 shows the SDS PAGE analysis of A2 Beta Casein clones. The results show is showing targeted protein bands around 25 kda.

### Western Blot

Day 1: SDS-PAGE: 10-12% separating gel

Equilibrate the membrane in transfer buffer for 15 minutes. Transfer of proteins to membrane for 3 hours (50 V) in cooling condition. After the transfer allow the membrane to dry (For fixation of proteins on membrane). Incubate the blot overnight in blocking buffer at 4° C. without shaking.
Day 2: Next day, immerse the blot in primary antibody. Incubate the blot for 24 hours at 4° C. without shaking.
Day 3: Next day wash the blot with TBS buffer three time for 5 minutes each. Incubate the membrane with secondary antibody for 1 hour with shaking at room temperature. Again, wash the blot for three times with TBS buffer 10 minutes each (Give first wash with TBST, remaining two washes with TBS). Incubate the blot with substrate until the dark bands are observed.

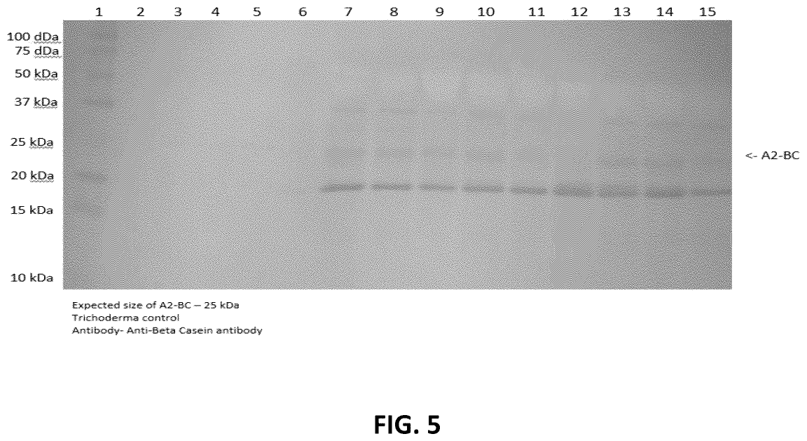

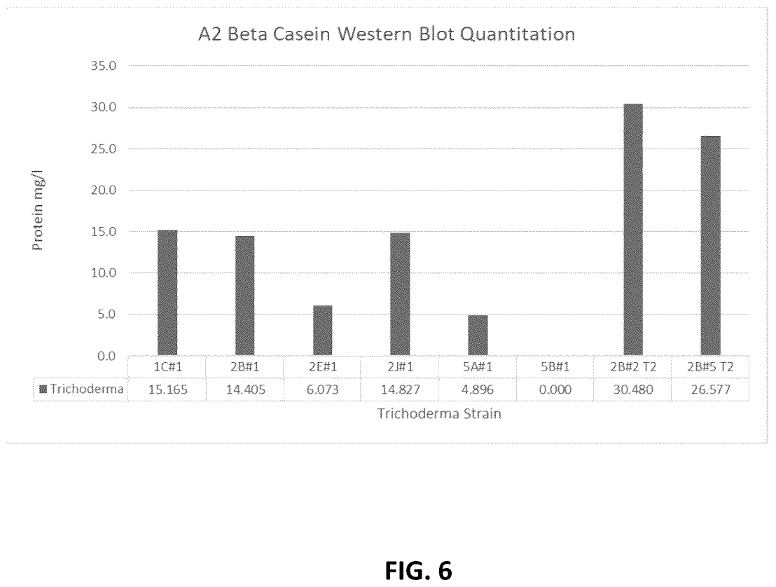

FIG. 5 shows Western blot analysis of A2 Beta Casein clones. The expression of target protein confirmed by Western blot. FIG. 6 shows A2 Beta Casein Quantitation which shows an initial titre of targeted A2 beta casein in 30 mg/L.

Thus, after obtaining the best strains after centrifuging the broth, the inventors performed downstream process to isolate and purify the target milk protein.

The foregoing broadly outlines the features and technical advantages of the present disclosure in order that the detailed description of the disclosure that follows may be better understood. It should be appreciated by those skilled in the art that the conception and specific embodiment disclosed may be readily utilized as a basis for modifying the disclosed methods or for carrying out the same purposes of the present disclosure.

It must be noted that as used herein, the singular forms “a”, “an”, and “the”, include plural references unless the context clearly indicates otherwise. Thus, for example, reference to “an expression cassette” includes one or more of the expression cassettes disclosed herein and reference to “the method” includes reference to equivalent steps and methods known to those of ordinary skill in the art that could be modified or substituted for the methods described herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

The methods and techniques of the present invention are generally performed according to conventional methods well-known in the art and as described in various general and more specific references that are cited and discussed throughout the present specification unless otherwise indicated. See, e. g., Sambrook et al., Molecular Cloning: A Laboratory Manual, 3rd ed., Cold Spring Harbor Laboratory Press, Cold Spring Harbor, N. Y. (2001); Ausubel et al., Current Protocols in Molecular Biology, J, Greene Publishing Associates (1992, and Supplements to 2002); Handbook of Biochemistry: Section A Proteins, Vol I 1976 CRC Press; Handbook of Biochemistry: Section A Proteins, Vol II 1976 CRC Press. The nomenclatures used in connection with, and the laboratory procedures and techniques of, molecular and cellular biology, protein biochemistry, enzymology and medicinal and pharmaceutical chemistry described herein are those well-known and commonly used in the art.

We claim:

1. A recombinant vector system for the production of animal free milk proteins comprising a polynucleotide sequence encoding a polypeptide sequence encoding the animal free milk protein selected from the group comprising of Beta-casein, Kappa-casein, Alpha-S1-casein, Alpha-S2-casein, Alpha-lactalbumin, Beta-lactoglobulin, Lactoferrin, and/or Transferrin, a nutritional selection marker polynucleotide sequence operably linked to a promoter, a nucleotide sequence encoding a ‘HIS’ tag, a nucleotide sequence encoding a bacterial resistance marker and a transcription terminator.

2. The recombinant vector as claimed in claim 1, wherein the animal free milk protein is Beta-casein.

3. The recombinant vector as claimed in claim 1, wherein the animal free milk protein is A2 Beta-casein.

4. The recombinant vector as claimed in claim 1, wherein the animal free milk protein is encoded by a polypeptide sequence having 90% similarity to SEQ ID No. 1.

5. The recombinant vector as claimed in claim 1, wherein the vector system comprises:
a. a nucleotide sequence encoding a promoter selected from T. reesei cellobiohydrolase 1 (cbh1) promoter, or T. reesei Ppdc1 promoter or, T. reesei PamdS promoter, or T. reesei Psdh promoter;
b. a nucleotide sequence encoding signal peptide selected from CBH1 signal peptide, or SPcbh1 signal peptide;
c. a nucleotide sequence encoding CBH1 core coding sequence,
d. a nucleotide sequence encoding a flexible linker,
e. a nucleotide sequence encoding a Kex2 protease recognition/cleavage site,
f. a nucleotide sequence encoding an animal free milk protein having 90% similarity to SEQ ID No. 2,
g. a nucleotide sequence encoding a ‘HIS’ tag;
h. a nucleotide sequence encoding a terminator sequence selected from T. viride cbh1 terminator, or Trichoderma reesei Tcbh1 terminator, or Trichoderma reesei Tpdc1 terminator, or Trichoderma reesei TamdS terminator, or Trichoderma reesei Tsdh terminator;
and
i. a nucleotide sequence encoding an acetamidase gene (amdS), or succinate Dehydrogenase Subunit Genes (sdh), or an hygromycin phosphotransferase (hph) gene.

6. The recombinant vector as claimed in claim 1, wherein the vector system comprises:
a. a nucleotide sequence encoding T. reesei cellobiohydrolase 1 (cbh1) promoter,
b. a nucleotide sequence encoding CBH1 signal peptide,
c. a nucleotide sequence encoding CBH1 core coding sequence,
d. a nucleotide sequence encoding a flexible linker,
e. a nucleotide sequence encoding a Kex2 protease recognition/cleavage site,
f. nucleotide sequence encoding an animal free milk protein having 90% similarity to SEQ ID No. 2,
g. a nucleotide sequence encoding a ‘HIS’ tag;
h. a nucleotide sequence encoding T. viride cbh1 terminator, and
i. a nucleotide sequence encoding an acetamidase gene (amdS).

7. The recombinant vector as claimed in claim 1, wherein the animal-free milk protein mimics the naturally occurring milk proteins.

8. The recombinant vector as claimed in claim 1, wherein the animal-free milk protein wherein the protein is obtained after codon optimization.

9. A method for the production of animal-free milk proteins in recombinant host cells, the method comprising the steps of:
i. designing plasmid expression vectors comprising a polynucleotide sequence encoding a polypeptide sequence encoding the animal free milk protein having 90% similarity to SEQ ID No. 1 operably linked to promoter by synthesis of gene segments and multiplication of gene segments using PCR techniques;
ii. carrying spore isolation and protoplastation of fungal host cell to obtain the competent protoplast;
iii. combining the recombinant vector with the competent protoplast obtained in step (ii) on ice followed by incubating the nutrient media plate for 2-4 weeks; and
iv. isolating the sporulating clones obtained in step (iii) by analysing the genomic DNA to identify the overexpressing milk protein gene;
v. inoculating the positive transformed clones identified in step (iv) in PDB media in baffled conical flask and growing them in a shaker at 150-180 rpm at 28-30° C. for 5-7 days;
vi. collecting the supernatant from the samples cultured in step (v) every 24 hr time interval centrifugating at 5000-8000 rpm for 5 min at 2-4° C. to harvest the animal free milk proteins;
vii. analysing the supernatant using SDS-PAGE and western blot to confirm the overexpression of beta-casein, followed by isolation and purification of the animal free milk protein.

10. The method as claimed in claim 9, wherein step (ii) of the method comprises the following steps:
a. adding double autoclaved Milli-Q water in the Trichoderma reesei RUT-C30 plate or slant to rinse the slant/plate surface to release the spores from the mycelium;
b. filtering the suspension obtained in step (a) through autoclaved miracloth in the laminar to collect filtrate containing spores or conidia;
c. washing the spore suspension by centrifuge at 5000-8000 rpm and 1-5 mins and discarding the supernatant followed by washing spore pellet with Milli-Q water and centrifuging at 5000 rpm for 3 min,
d. repeating this washing step 3 times and re-suspending the spore pellet in autoclaved Milli-Q water;
e. inoculating spore suspension in PDB and incubating at 25° C. to 28° C. with 100 to 200 rpm for 5 to 6 hrs followed by confirming the spore germination under microscope to carry out protoplastation;
f. centrifuging the spore suspension obtained in step (e) at 5000-8000 rpm and 3-5 mins to obtain the pellet and discarding the supernatant;
g. adding SMC to the pellet and centrifuging at 5000-8000 rpm and 5-8 mins, repeated this step four times;
h. incubating the spore pellet with lysing enzymes for 30 min-1 hour with regular gentle tapping/mixing till protoplastation is shown on 60% population,
i. adding SMC solution to the suspension and centrifuging at 2000-8000 rpm and 3-5 mins and discarding the supernatant, this step is repeated thrice with SMC;
j. Wash the pellet obtained in step (i) with STC buffer and centrifuging at 2000-8000 rpm and 3-5 mins and discard supernatant followed by dissolving the pellet in STC to obtain the competent protoplast.

11. The method as claimed in claim 9, wherein step (iv) of the method comprises the following steps:
a. Growing transformed host cells in MMS-N+Acetamide liquid media at 28-30° C. for 5 to 10 days under constant shaking conditions;
b. centrifuging the culture in a tube at 12000×g to 18000×g for 15-20 mins followed by discarding the supernatant using a pipette and collecting the cell pellet and freezing it using Liquid N2¬ for storing at −80° C. to crush the genomic DNA in the cell pellets in liquid N2¬;
c. adding lysis buffer to crushed cell pellet followed by subjecting the samples to vortex several times and adding Phenol:Chloroform:Isoamyl alcohol mixture to vortexed sample;
d. centrifuging the lysed cell suspension obtained in step (c) at 12000×g to 18000×g for 15-20 min at room temperature and collecting the upper aqueous layer in a microcentrifuge tube, and adding to it, 100% ethanol followed by mixing the resulting solution by inverting the tube several times;
e. centrifuging the solution obtained in step (d) at 12000×g to 18000×g for 5 to 10 min at room temperature, discarding the supernatant and drying the DNA pellet at 37° C. incubator;
f. washing the dried DNA pellet with 70% Ethanol and centrifugating at 12000×g to 18000×g for 5-10 min followed by drying the resulting pellet at 37° C. and resuspended in elution buffer supplemented with of RNaseA and incubating in a 50-60° C. heat block for 10-20 min to obtain the genomic DNA.

12. The method as claimed in claim 9, wherein the host cell is selected from bacterial, yeast or fungal cells.

13. The method as claimed in claim 9, wherein the host cell is a fungal cell.

14. The method as claimed in claim 9, wherein the host cell is Trichoderma reseei.

15. A composition comprising the animal free milk protein along with pharmaceutically or nutraceutically acceptable carriers or vehicles.

16. A nucleic acid sequence encoding the recombinant fusion protein encoded by the recombinant polynucleotide sequence having 90% similarity to SEQ ID No. 2.

17. A recombinant host cell comprising recombinant vector comprising the polynucleotide sequence encoding the polypeptide sequence having 90% similarity to SEQ ID No. 1 as claimed in claim 1.

18. The host cell as claimed in claim 17, wherein the host cell is selected from bacterial, plant, yeast or fungal cells.

19. The host cell as claimed in claim 17, wherein the host cell is a fungal cell.

20. A food composition comprising an animal free milk protein having the amino acid sequence having 90% similarity to SEQ ID No. 1 as claimed in claim 1, wherein the food composition is selected from the group consisting of cheese and processed cheese products, yogurt and fermented dairy products, directly acidified counterparts of fermented dairy products, cottage cheese dressing, frozen dairy products, frozen desserts, desserts, baked goods, toppings, icings, fillings, low-fat spreads, dairy-based dry mixes, soups, sauces, salad dressing, geriatric nutrition, creams and creamers, analog dairy products, follow-up formula, baby formula, infant formula, milk, dairy beverages, acid dairy drinks, dairy substitutes, smoothies, milk tea, butter, margarine, butter alternatives, growing up milks, low-lactose products and beverages, medical and clinical nutrition products, protein/nutrition bar applications, sports beverages, confections, meat products, analog meat products, meal replacement beverages, weight management food and beverages, cultured buttermilk, sour cream, yogurt, skyr, leben, lassi, kefir, powder containing a milk protein, and low-lactose products.
